(19) United States
(12) Patent Application Publication
(10) Pub. No.: US 2010/0217321 A1
(43) Pub. Date: Aug. 26, 2010

(54) SPACING MEANS FOR INSERTION BETWEEN SPINOUS PROCESSES OF ADJACENT VERTEBRAE

(75) Inventors: Geoffrey Harrison Galley, London (GB); James Bernard Allibone, Radlett (GB); Mohammed Hamza Hilali Noordeen, London (GB); Tring Anthony Taylor, Tring (GB); Stewart Kenneth Tucker, London (GB)

Correspondence Address: HARNESS, DICKEY & PIERCE, P.L.C., P.O. BOX 828, BLOOMFIELD HILLS, MI, 48303, US

(73) Assignee: EBI, L.L.C., Parsippanny, NJ (US)

(21) Appl. No.: 12/294,378

(22) PCT Filed: Mar. 23, 2007
(86) PCT No.: PCT/US07/07243

§ 371 Date: Apr. 20, 2010

(30) Foreign Application Priority Data
Mar. 24, 2006 (GB) 0605962.0

(51) Int. Cl. A61B 17/70

(52) U.S. Cl. 606/249

(57) ABSTRACT

A device for insertion between the spinous processes of adjacent vertebrae of the human spine includes a first tapered screw-like component dismountably assembled to a second component by means of a centrally located shaped rod. The second component includes a self-locating spacer member which remains in position between adjacent spinous processes after removal of the tapered screw-like portion.

This invention relates to the insertion of one or more spacing means in the human vertebral column and is an improved means of provision and insertion off such spacing means. Recent advances in minimally invasive spinal surgery have led to the adoption of spacing means in order to increase the distance between adjacent spinous processes extending from the rear of the spinal vertebrae. Such spacing means are presently marketed by U.S. companies such as Medtronics Inc. and Saint Francis Medical Inc. and details of such means are show in the attached documents B1 and B2 which are copies of the brochures of the above mentioned companies relating to these products.

The present spacing means each suffer a distinct disadvantage in that due to their size and other considerations neither of them is suitable for use in the cervical spine. Further the said spacing means each require the creation of a significant surgical trauma in order to provide access for the insertion of the said spacing means into the human body. It is an object of the present invention to provide a spacing means which can be positioned between the spinous processes using only a minimally invasive surgical procedure thereby reducing the degree of trauma suffered by the patient and hence the time required for the healing of the insertion wound.

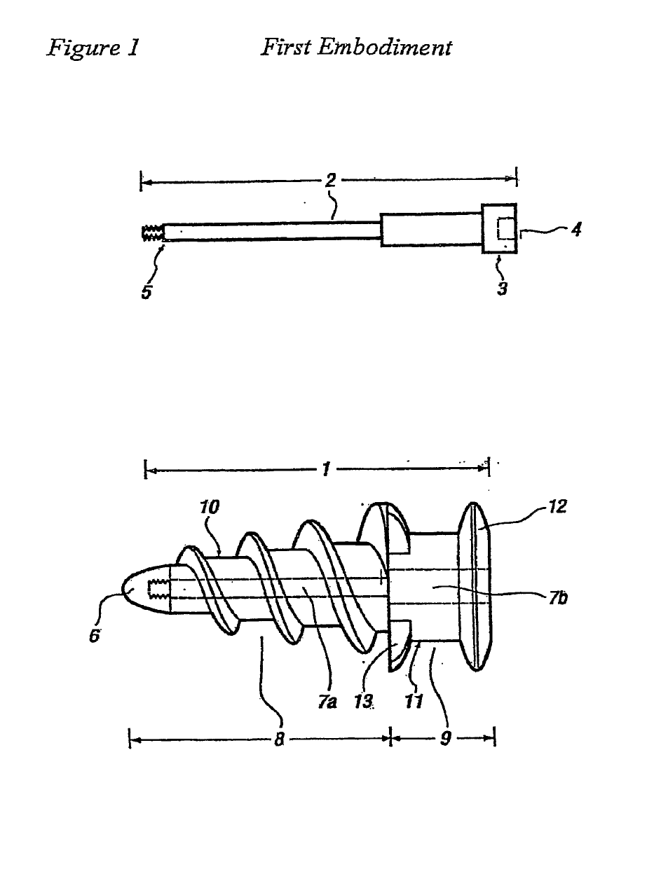

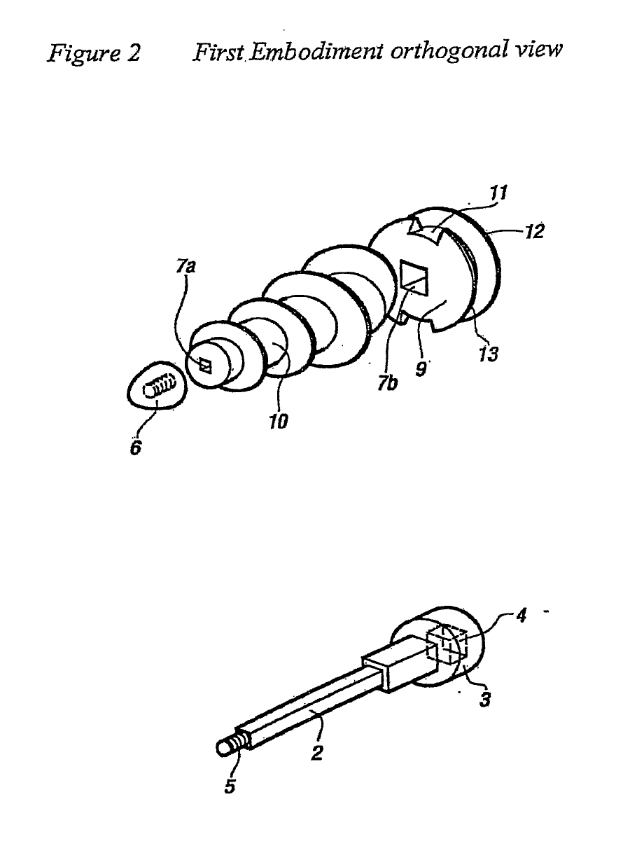

The present invention is described by reference to FIG. 1 and FIG. 2 which respectively represent a plan view and an orthogonal view of a first embodiment of the device of the invention. The device consists of a spacing means in the form of a segmented screw-like assembly (1) which is held together by means of a shaped rod (2). One end of said shaped rod (2) is provided with a concentric cap (3) having a recess (4) into which a tool may be inserted in order to rotate the said shaped rod. The other end of the said shaped rod is provided with a screwed portion (5), onto which a threaded shaped “blind” nut (6) may be screwed. The said shaped rod (2) is formed with a square cross-section so as to engage with similarly shaped holes (7a and 7b) provided through the center of the portions of the device. The cross-sectional dimensions of the length of the said shaped rod engaging with the distinct portions (8) & (9) of the device are different so as to provide for the separate rotation of different portions of the device.

The first portion (8) of the said first embodiment, of the device is comprised of one or more components, which singly or together form a section of a tapered screw the root of which (10) may be advantageously disposed more or less parallel to the longitudinal axis of the device and which may be of a form (not shown) appropriate for the location of one or more spinous processes which may rest on the said root.

The second portion (9) of the said first embodiment of the device comprises a spacing member which will be inserted between the said processes of the said spinal vertebrae during the required surgical procedure and which will remain in position upon the completion of the surgical procedure. The said second portion comprises a central hub (11) of a form which is adapted so as to provide a surface upon which the said spinous processes may bear when the said second portion rests between two such spinous processes. The cross-sectional profile of the said hub, which lies at right angles to the horizontal axis through the center of the component may vary along the direction of the said horizontal axis so as to provide lateral location for the spinous processes, which rest against it (not shown).

As can be seen in FIGS. 1 & 2 the hub section (11) of the second portion of the said first embodiment of the device is provided at either end with a terminating annulus (12) or part annulus (13). The said part annulus may be orientated (not shown) so as to assist the forward movement of the said second portion of the first embodiment of the device as the assembled device is rotated about its axis in engagement with the said spinous processes and their surrounding tissue.

In order to effect the insertion of the spacing member (9) the surgeon first provides access to the left and right hand sides of the adjacent spinous processes between which it is desired to insert the said spacing member using minimally invasive techniques known to those skilled in the surgical art. The assembled first embodiment of the device is inserted into the area between the adjacent spinous processes and rotated using a driving tool (not shown) which may be advantageously fitted with a ratchet device for ease of operation. As the said first embodiment of the device advances through the space between the adjacent spinous processes the increasing cross-sectional dimensions of the root of the first portion of the device causes the adjacent spinous processes to progressively separate.

The said second component (9) of the first embodiment of the device is selected by the surgeon to provide the desired spacing between the adjacent spinous processes upon completion of the surgical procedure. When the said second component of the device enters the space between the adjacent spinous processes and has been set at the appropriate rotational angle, the leading end of the first portion of the screw-like device may be accessed on the left-hand side of the spinous processes. The shaped nut (6) which has been screwed on to the shaped rod (2) during the assembly of the device may now be gripped using a surgical pliers and rotated so as to remove it from the screwed end of the shaped rod. Alternatively the shaped nut (6) may be provided with a means of attaching a special-purpose tool (not shown) which may in turn be provided with a ratchet for ease of operation in removing the shaped nut from the said shaped rod.

Following the removal of the shaped nut (6) from the shaped rod (2), the shaped rod is withdrawn by the surgeon from the right hand side of the spinous processes. At this point, a second shaped rod (not shown) having cross-sectional dimensions similar to those of the said square section hole (7a) in the said first component of the first embodiment of the device, but smaller than those of the square section hole (7b) in the said second component (9) of the device (alternatively referred to as “the spacing member”) is inserted from the right hand side of the spinous processes and fed through the larger square hole (7b) in the said spacing member (9) so as to pass through the smaller square section hole (7a) in the said first component of the first embodiment of the device. The shaped nut (6) is now screwed onto the threaded end of the said second shaped rod and the first component (8) of the device is now rotated by means of the smaller shaped rod so as to drive it forward, releasing it from the patient's body to the left of the spinous processes. The said shaped nut (6) is now unscrewed from the said second shaped rod and the rod is withdrawn from the body of the patient on the right hand side of the spinous processes leaving the said second component (or spacing member) (9) of the said segmented tapered screw assembly disposed between the spinous processes of the required adjacent vertebrae. In performing the surgical procedure the insertion of the spacing member may, if desired, be made from the left hand side of the spinous processes in which event the instructions as to left hand and right hand positioning are interchanged. The spacing member (9) may, if desired, be provided with an attached clip or suture (not shown) by means of which it may be secured to the adjacent spinous processes in order to prevent dislodgement.

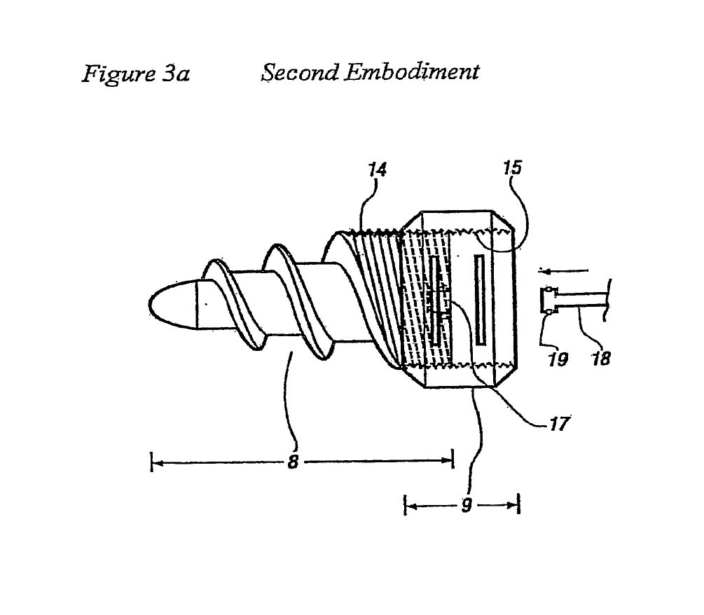

In a second and preferred embodiment of the device shown in FIG. 3a, the first component (8) of the device is provided with a threaded portion (14) which engages with the internally threaded bore (15) of the second component (9) of the device otherwise again referred to as “the spacing member”. The said second component (9) may be advantageously provided with a means (not shown) of limiting the extent to which the first portion may travel along the bore of the second component and project beyond the end of the second component. The second component is further provided with four or more slots (16) through which sprung members may project upon final disposition of the device as shown in FIG. 3b at (1).

The assembled device is inserted using a shaped rod the end portion of which is shown at (18 in FIG. 3a) which may advantageously be of a flexible nature. The said shaped rod locks into a recess (17) in the first component (8) of the second embodiment of the device by means of a sprung ball fitting at its end (19). Following the positioning of the spacing member (9) the first component of the device (8) is withdrawn down the screwed bore (15) of the said spacing member. Clips (20 in FIG. 3b) are then entered into the open bore of the second component using a compressing tool (not shown) which releases the clips within the bore to project through the slots provided in the spacing member (9).

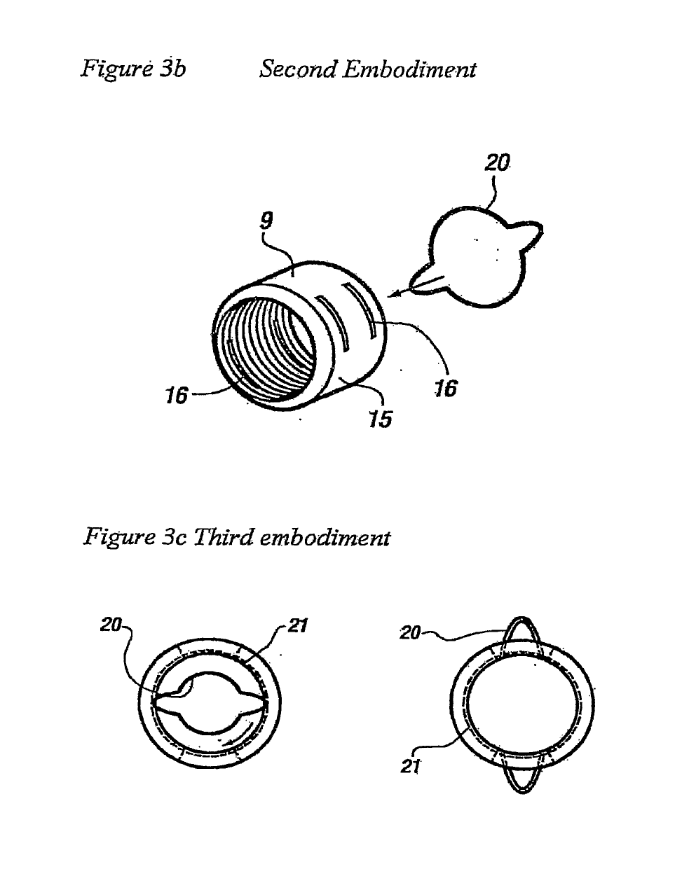

In a further embodiment shown in FIG. 3b, the clips (20) are disposed within the bore of the second component behind the extended first component upon insertion of the assembled device. After positioning of the spacing member between the spinous processes in the manner described above with reference to FIG. 5a, the clips are repositioned to project through the slots with the non-projecting portion of the clips seated in a housing groove 21 set in the screwed internal surface of the bore of the spacing member as shown in FIG. 3c. The first component is then screwed back through the bore of the said spacing member and withdrawn from the body of the patient as described in relation to the second embodiment of the device but in this case after passing over the seated portions of the locating clips.

The device of the invention may be fabricated from any material, which is suitable for use in the human body. The preferred material for first and second components as well as the blind nut may be any plastic material, which has been formulated for use in the human body. The said plastic material may be coated or treated with other material in order to render it more compatible with surrounding tissue. The spring clips referred to in relation to second and third embodiments may be advantageously formed from stainless spring steel or any suitable metal alloy.

In order to facilitate viewing of the device during insertion in the human body, the material of the said flexible enclosure may be rendered radio-opaque by inclusion therein of a radio opaque material in forms familiar to those skilled in the art of radiographic imaging.

The device of the invention may be manufactured in a variety of sizes suitable for insertion at different points in the spinal column. First and second components of different sizes may be combined to achieve the best result in any given location. The device of the invention has the advantage of entitling the necessary independent displacement of the adjacent vertebrae to the required separation, which is achieved automatically upon the positioning of the spacing member of the device between the adjacent spinous processes. It is thus unnecessary to increase the separation of the adjacent vertebrae beyond the desired separation in order to facilitate the insertion of the spacing member.

1-8. (canceled)

9. A device for insertion between adjacent spinous processes of adjacent vertebrae comprising:
a screw having a body that tapers from a first end to a second end, with at least one thread formed on an exterior surface of the body to enable the screw to pass through an anatomy, the taper of the screw adapted to progressively separate the adjacent spinous processes;
a cylindrical spacer defining a bore that removably couples the spacer to the screw, the spacer adapted to support the adjacent spinous processes; and
wherein the screw is uncoupled from the spacer after the spacer is positioned between the adjacent spinous processes such that the spacer remains between the adjacent vertebrae.

10. The device of claim 9, further comprising:
a rod having a threaded end;
a cap opposite the threaded end; and
an intermediate section separating the threaded end from the cap;
wherein the body of the screw defines a bore that receives a first portion of the intermediate section of the rod, with the screw sized so that the threaded end of the rod extends beyond an end of the screw.

11. The device of claim 10, wherein the spacer defines a bore that receives a second portion of the intermediate section of the rod and the cap of the rod therein so that the spacer is coupled adjacent to the screw by the rod.

12. The device of claim 11, further comprising:
a nut that threadably engages the threaded end of the rod so that the rod is disposed entirely within the nut, body of the screw and the spacer; and
wherein the intermediate section of the rod is sized so that the rod passes through the body of the screw upon removal of the nut from the threaded end.

13. The device of claim 10, wherein the cap is concentric with the rod and defines a recess adapted to enable a driver to rotate the rod.

14. The device of claim 11, wherein the intermediate section of the rod has a square cross-section to enable the body of the screw and the spacer to rotate with the rod.

15. The device of claim 9, wherein the spacer further comprises:
a first formed hub that is adapted to support a respective one of the adjacent spinous processes; and
a second formed hub opposite the first formed hub and adapted to support the other of the adjacent spinous processes.

16. The device of claim 15, wherein the spacer further comprises:
a first annulus extending from the spacer;
a second annulus extending from the spacer, the second annulus spaced apart from the first annulus and positioned adjacent to the screw when the screw and spacer are coupled to the rod; and
wherein the first hub and the second hub are formed through the second annulus.

17. The device of claim 12, wherein upon removal of the nut, the rod and the screw are removable from the anatomy such that the spacer remains disposed between the adjacent spinous processes.

18. The device of claim 9, wherein the body of the screw includes a recess adapted to engage a driver operable to drive the screw into the anatomy.

19. The device of claim 18, wherein the spacer defines a throughbore that includes a plurality of threads that mate with a second plurality of threads formed on the body of the screw to removably couple the spacer to the screw.

20. The device of claim 19, further comprising:
at least one slot formed along a circumference of the spacer; and
at least one clip including at least one projection that engages the at least one slot to secure the spacer in the anatomy.

21. The device of claim 20, wherein a compression tool is used to compress the at least one clip so that the at least one clip fits into the throughbore of the spacer, and the release of the at least one clip by the compression tool biases the at least one projection into engagement with the at least one slot.

22. The device of claim 18, wherein the driver is a shaped rod and includes a sprung ball fitting at one end that engages the recess to enable the driver to drive the screw into the anatomy.

23. The device of claim 20, wherein four slots are formed along the circumference of the spacer, with two of the slots formed opposite the other two of the slots, and the at least one clip includes at least two projections formed on opposite sides of the at least one clip.

24. A device for insertion between adjacent spinous processes of adjacent vertebrae comprising:
a screw having a body that tapers from a first end to a second end, with at least one thread formed on an exterior surface of the body to enable the screw to pass through an anatomy, the taper of the screw adapted to progressively separate the adjacent spinous processes;
a cylindrical spacer that defines a bore that removably couples the spacer to the screw, the spacer including a first formed hub and a second formed hub opposite the first formed hub; and
wherein the screw is uncoupled from the spacer after the spacer is positioned between the adjacent spinous processes so that the first formed hub supports a respective one of the adjacent spinous processes and the second formed hub supports the other of the adjacent spinous processes.

25. The device of claim 24, further comprising:
a rod having a threaded end;
a cap opposite the threaded end; and
an intermediate section separating the threaded end from the cap, the intermediate section having a first portion and a second portion;
wherein the body of the screw defines a bore that receives the first portion of the intermediate section, with the screw sized so that the threaded end of the rod extends beyond an end of the screw, and the bore of the spacer receives the second portion of the intermediate section and the cap of the rod therein so that the spacer is coupled adjacent to the screw by the rod.

26. The device of claim 25, further comprising:
a nut that threadably engages the threaded end of the rod so that the rod is disposed entirely within the nut, body of the screw and the spacer; and
wherein the intermediate section of the rod is sized so that the rod passes through the body of the screw so that the rod and the screw are removable from the anatomy upon removal of the nut from the threaded end.

27. The device of claim 26, wherein the intermediate section of the rod has a square cross-section to enable the body of the screw and the spacer to rotate with the rod.

28. A device for insertion between adjacent spinous processes of adjacent vertebrae comprising:
a screw having a body that tapers from a first end to a second end, with at least one thread formed on an exterior surface of the body to enable the screw to pass through an anatomy, the taper of the screw adapted to progressively separate the adjacent spinous processes, the body of the screw defining a bore;
a cylindrical spacer that defines a bore that removably couples the spacer to the screw, the spacer including a first formed hub that is adapted to support a respective one of the adjacent spinous processes and a second formed hub opposite the first formed hub that is adapted to support the other of the adjacent spinous processes;
a rod having a threaded end that extends beyond an end of the screw, with a cap opposite the threaded end and an intermediate section that separates the threaded end from the cap, the intermediate section having a first portion that is received within the bore of the screw and a second portion that is received within the bore of the spacer so that the spacer is coupled adjacent to the screw by the rod, the intermediate portion of the rod having a square cross-section to enable the body of the screw and the spacer to rotate with the rod;
a nut that threadably engages the threaded end of the rod so that the rod is disposed entirely within the nut, body of the screw and the spacer; and
wherein the nut is removable after the spacer is positioned between the adjacent spinous processes to uncouple the screw from the spacer so that the rod and the screw are removable from the anatomy such that the spacer remains disposed between the adjacent spinous processes.
